(19) United States
(12) Patent Application Publication
(10) Pub. No.: US 2010/0112122 A1
(43) Pub. Date: May 6, 2010

(54) METHOD OF PREFERENTIALLY REDUCING ABSORBABILITY OF SATURATED FATTY ACIDS

(76) Inventors: Yun-Jeong Hong, Berkeley, CA (US); Scott A. Young, Midland, MI (US); Maciej Turowski, Midland, MI (US); Wallace H. Yokoyama, Berkeley, CA (US); Marsha L. Langhorst, Midland, MI (US); David R. Albers, Midland, MI (US)

Correspondence Address: The Dow Chemical Company, Intellectual Property Section, P.O. Box 1967, Midland, MI, 48641-1967, US

(21) Appl. No.: 12/112,775

(22) Filed: Apr. 30, 2008

(51) Int. Cl. A23K 1/16; A23D 9/00; A23D 7/00; A23L 1/39; G01N 33/03

(52) U.S. Cl. 426/2; 426/601; 426/580; 426/606; 426/589; 426/231

(57) ABSTRACT

A method of reducing the amount of saturated fatty acids capable of being absorbed by an animal body relative to the amount of unsaturated fatty acids capable of being absorbed by an animal body in or from a fat-containing food product comprises the steps of
i) identifying a food product comprising one or more saturated fatty acids and one or more unsaturated fatty acids, and
ii) incorporating one or more water-soluble cellulose derivatives in the food product or administering one or more water-soluble cellulose derivatives before, during or after the consumption of the food product.

### FIELD OF THE INVENTION

The present invention relates to a method of reducing the amount of saturated fatty acids capable of being absorbed by an animal body relative to the amount of unsaturated fatty acids capable of being absorbed by an animal body.

### BACKGROUND OF THE INVENTION

Fats provide a highly concentrated source of energy in food products. Although fats are essential for functioning of the body, they are also a major contributor of calories in the modern diet that in many cases leads to people becoming overweight or even obese. Since overweight or obesity has become a serious problem in many societies, huge research efforts are undertaken to find ways of reducing this problem.

One way of addressing the problem is helping people to reduce their food caloric intake. Soluble dietary fibers forming viscous solutions water, i.e. guar gum and methyl cellulose, have previously shown to be efficient to a certain extent at decreasing energy intake and weight loss in obese subjects. Studies of soluble dietary fibers have been published by E. Evans & D. S. Miller, Bulking Agents in the Treatment of Obesity, Nutr. Metabol. 1975, 18, 199-203; J. N. Badham, Methylcellulose for Obesity, The Lancet 1953, p. 1316; J. Yudkin, The Causes and Cure of Obesity, The Lancet 1959, Special Articles, pp. 1135-1137; and M. L. Durrant & P. Royston, The Effect Of Preloads Of Varying Energy Density And Methyl Cellulose On Hunger, Appetite And Salivation, Proceedings of the Nutrition Society 1978, Vol. 37, p. 87A.

Without wanting to be bound by the theory, Applicants believe that the soluble dietary fibers slow gastric emptying and maintain a feeling of satiety over a longer time period thereby reducing the food caloric intake.

Non-starch polysaccharides which swell when placed in aqueous solutions because of their strongly hydrophilic properties have been known to be effective bulk laxatives for a long time. Cellulose ethers, such as methylcellulose and carboxymethylcellulose have been taught as effective bulk laxative aids. Their mechanism of action involves increasing both the water content and the bulk content of the stool, as well as lubricating the stool; thereby relieving constipation.

U.S. Pat. No. 5,462,742 discloses a dietary fiber composition comprising a water-soluble nonionic cellulose ether having a cloud point not higher than 35° C. in combination with a charged surfactant and optional additives in water. The dietary fiber composition is a liquid solution at room temperature and a gel in the gastrointestinal tract at body temperature.

The International Patent Application published as WO 03/053451 discloses that resorption of plant fats from the gastro-intestinal tract is reduced by a gelling mechanism when an aqueous solution of an ionic or non-ionic cellulose ether is consumed together with a meal. Increased fat excretion is detected.

Chitosan (1-4-β-D-polyglucosamine) has become popular as a fat binder, binding dietary fats in vivo and thus rendering them nutritionally unavailable. The bound fats are excreted instead of being absorbed or utilized. The International Patent Application published as WO 03/097714 gives an overview on the effects on Chitosan and discloses the use of inter-polymer complexes of glucosamine and polyacrylic acid for fat binding. On the other hand, Chitosan's ability to bind fat is challenged in other publications, such as M. D. Gades & J. S. Stern, Chitosan Supplementation and Fecal Fat Excretion in Men, Obesity Research 2003, 11, 683-688.

The effects of various dietary fibers or their likenesses on the apparent fat digestibility by rats fed on a high-fat diet is published by Deuchi et al., Decreasing Effect of Chitosan on the Apparent Fat Digestibility by Rats Fed on a High-fat Diet, Bioscience, Biotechnology, and Biochemistry (ISSN 0916-8451), 1994, vol. 58, no 9, pp. 1613-1616. When compared with cellulose (control), 10 of the 23 tested fibers significantly increased the fecal lipid excretion. Among these fibers, Chitosan markedly increased the fecal lipid excretion and reduced the apparent fat digestibility to about a half relative to the control. The apparent protein digestibility was not greatly affected by Chitosan. The fatty acid composition of the fecal lipids closely reflected that of the dietary fat.

The International Patent Application published as WO 00/13667 discloses that Orlistat is a potent and selective inhibitor of pancreatic lipase. The patent application discloses that lipase inhibitors have side-effect issues like fecal incontinence. To reduce such side effects, particularly to reduce stool liquidity, WO 00/13667 discloses a pharmaceutical composition which comprises a combination of a rapidly disintegrating methylcellulose and Orlistat in combination.

Fats are usually combinations of saturated and unsaturated fatty acids. Saturated fatty acids are typically found in animal products such as butter or lard, and in some vegetable oils, such as coconut, palm and palm kernel oils and are one of the causes of increase in low density lipoprotein (LDL), the so-called “bad cholesterol”. Unsaturated fatty acids are considered to be healthier, because they tend to contribute to lower blood cholesterol. Linoleic acid and alpha-linolenic acid are two unsaturated acids which cannot be made in the body from other substrates and must be supplied in the food. Therefore, they are designated as essential fatty acids. In many societies people consume much meat and dairy products which contain fats rich in saturated fatty acids. These diets generally lead to an over-supply of saturated fatty acids, but not to an oversupply of unsaturated fatty acids, and even less to an oversupply of essential fatty acids. Some skilled artisans even warn against a potential undersupply of essential fatty acids. Accordingly, it would be highly desirable to render saturated fatty acids more selectively nutritionally unavailable than unsaturated fatty acids.

It has been reported in the art that dietary fats consist primarily (over 90 percent) of triacylglycerides, which are composed of one glycerol molecule esterified with three fatty acid molecules, and minor amounts of phospholipids and sterols. Free fatty acids are hydrocarbon chains that contain a methyl (CH₃—) and a carboxyl (—COOH) end. The fatty acids vary in carbon chain length and degree of unsaturation (number of double bonds in the carbon chain). The fatty acids can be classified into the following categories: saturated fatty acids, monounsaturated fatty acids, and polyunsaturated fatty acids. Dietary fats originate from both animal and plant products. In general, animal fats have higher melting points and are solid at room temperature, which is a reflection of their high content of saturated fatty acids. Plant fats generally have lower melting points and are liquid at room temperature; this is explained by their high content of unsaturated fatty acids.

Dietary fats undergo lipolysis by lipases in the gastrointestinal tract prior to absorption. Although there are lipases in the saliva and gastric secretion, most lipolysis occurs in the small intestine. In general, triacylglycerides do not get absorbed into the intestine. The hydrolysis of triacylglycerides is achieved through the action of pancreatic lipase, which requires colipase, also secreted by the pancreas for activity. In the intestine, fats are emulsified with bile salts and phospholipid are secreted into the intestine, hydrolyzed by pancreatic enzymes, and almost completely absorbed. Pancreatic lipase has high specificity for sn-1 and sn-3 positions of dietary triacylglycerides, resulting in the release of fatty acids from sn-1 and sn-3 positions and 2-monacylglycerides. These products of digestion are absorbed into the enterocyte and the triacylglycerides are reassembled, largely via the 2-monoacylglyceride pathway.

The International Patent Application published as WO 2007/073543 discloses that saturated fats are preferentially bound by alpha and/or beta cyclodextrin. The patent application discusses the effect of chitosan, alpha-cyclodextrin and complexed alpha-cyclodextrin on fecal fat excretion in rats. Alpha-cyclodextrin added directly to the rat diet had no affect on the excretion of the unsaturated fatty acid triolein, whereas complexed alpha-cyclodextrin slightly but significantly increased it. Both alpha-cyclodextrin and complexed alpha-cyclodextrin increased the excretion of the saturated fatty acid tripalmitin. Chitosan, which is known to increase fecal fat excretion in rats, significantly increased excretion of both triolein and tripalmitin, but in approximately equal proportions. Although alpha and/or beta cyclodextrin are reported to be very effective in enabling preferential excretion of saturated fatty acids over unsaturated fatty acids, one single type of compounds evidently cannot satisfy the need of all people for whom preferential excretion of saturated fatty acids over unsaturated fatty acids would be desirable to make the saturated fatty acids more selectively nutritionally unavailable than unsaturated fatty acids.

Accordingly it would be highly desirable to find another method of reducing the amount of saturated fat capable of being absorbed by an animal body relative to the amount of unsaturated fat capable of being absorbed by an animal body.

### SUMMARY OF THE INVENTION

One aspect of the present invention is a food product which comprises

a) a fat rich in one or more saturated fatty acids,
b) a fat rich in one or more unsaturated fatty acids, and
c) at least 2 weight percent of one or more water-soluble cellulose derivatives, based on the total weight of the food product.

Another aspect of the present invention is a food product in the form of a processed meat product, a dairy product, a fried product or a product able to be fried comprising i) one or more saturated fatty acids, ii) one or more unsaturated fatty acids, and iii) at least 2 weight percent of one or more water-soluble cellulose derivatives, based on the total weight of the food product.

Yet another aspect of the present invention is a method of reducing the amount of saturated fatty acids capable of being absorbed by an animal body relative to the amount of unsaturated fatty acids capable of being absorbed by an animal body in or from a fat-containing food product, the method comprising the steps of i) identifying a food product comprising one or more saturated fatty acids and one or more unsaturated fatty acids, and ii) incorporating one or more water-soluble cellulose derivatives in the food product or administering one or more water-soluble cellulose derivatives before, during or after the consumption of the food product.

Yet another aspect of the present invention is a method of reducing the effective caloric content contributed by one or more saturated fatty acids relative to the caloric content contributed by one or more unsaturated fatty acids in or from a fat-containing food product, the method comprising the steps of i) identifying a food product comprising one or more saturated fatty acids and one or more unsaturated fatty acids, and ii) incorporating one or more water-soluble cellulose derivatives in the food product or administering one or more water-soluble cellulose derivatives before, during or after the consumption of the food product.

Yet another aspect the present invention relates to a method of reducing the amount of predominantly saturated triacylglycerides capable of being hydrolyzed in the intestine by lipases relative to the amount of predominantly unsaturated triacylglycerides capable of being hydrolyzed in the intestine by lipases in or from a fat-containing food product comprising the steps of

i) identifying a food product comprising one or more predominantly saturated triacylglycerides and one or more predominantly unsaturated triacylglycerides, and
ii) incorporating one or more water-soluble cellulose derivatives in the food product or administering one or more water-soluble cellulose derivatives before, during or after the consumption of the food product.

### DETAILED DESCRIPTION OF THE INVENTION

It has been found that water-soluble cellulose derivatives, such as water-soluble cellulose ethers, are not only effective as bulk laxatives or for increasing excretion of plant fats. It has surprisingly been found that saturated fatty acids are preferentially excreted relative to the excretion of unsaturated fatty acids when a food product comprises one or more water-soluble cellulose derivatives besides saturated fatty acids and unsaturated fatty acids or when one or more water-soluble cellulose derivatives are administered before, during or after the consumption of a food product comprising saturated fatty acids and unsaturated fatty acids. The preferential excretion of saturated fatty acids is an indication that water-soluble cellulose derivatives are effective in preferentially reducing the amount of saturated fatty acids capable of being absorbed by an animal body relative to the amount of unsaturated fatty acids capable of being absorbed by an animal body in or from a food product that comprises one or more saturated fatty acids and one or more unsaturated fatty acids. This is highly advantageous and in contrast to the observations made by the skilled artisans evaluating chitosan.

In one aspect of the present invention the food product comprises a fat a) rich in one or more saturated fatty acids and a fat b) rich in one or more unsaturated fatty acids. The term “a fat a) rich in one or more saturated fatty acids” relates to a fat a) which originates from one or more sources and which is rich in one or more saturated fatty acids. The term “a fat b) rich in one or more unsaturated fatty acids” relates to a fat b) which originates from one or more sources and which is rich in one or more unsaturated fatty acids. The presence of one or more water-soluble cellulose derivatives in the food product or the administration of one or more water-soluble cellulose derivatives in combination with the food product is particularly effective for preferential excretion of saturated fatty acids relative to the excretion of unsaturated fatty acids if the food product comprises at least 1.5 weight percent, typically at least 2.5 weight percent, more typically at least 5 weight percent, and especially at least 10 weight percent of a fat rich in one or more saturated fatty acids and at least 1.5 weight percent, typically at least 2.5 weight percent, more typically at least 5 weight percent, and especially at least 7.5 weight percent of a fat rich in one or more unsaturated fatty acids. The preferred upper limit of a fat a) rich in one or more saturated fatty acids and a fat b) rich in one or more unsaturated fatty acids is mainly determined by nutricial and organoleptic considerations. Depending on the type of food, the preferred amount of total fats is typically up to about 85 weight percent, of which preferably 5 to 75 weight percent originate from a fat rich in one or more saturated fatty acids. The percentages are by total weight of the food product. The weight ratio between a) the fat rich in one or more saturated fatty acids and b) the fat rich in one or more unsaturated fatty acids is generally from 0.05 to 15:1, preferably from 0.1 to 15:1, more preferably from 0.15 to 1.5:1, and most preferably from 0.5 to 1.2:1. The term “fat” as used herein designates solid and liquid oils which essentially consist of one or more fatty acids or of which the major component is one or more fatty acids. The latter term means that generally at least 75 percent, preferably at least 85 percent, more preferably at least 95 percent of the fat consists of one or more fatty acids. Sources of fats include both animal and vegetable fats.

The term “fatty acids” as used herein includes free saturated and unsaturated fatty acids, monoglyceride, diglyceride and triglyceride esters of saturated and unsaturated fatty acids, phospholipids of saturated and unsaturated fatty acids, and cholesterol esters of saturated and unsaturated fatty acids. Triglyceride esters containing no or only one C═C double bond are predominantly saturated and are included in the term “saturated fatty acids”. Triglyceride esters containing two or more C═C double bonds are predominantly unsaturated and are included in the term “unsaturated fatty acids”.

Typically fats comprise more than one saturated, monounsaturated and/or polyunsaturated fatty acids. The fatty acids can be short chain fatty acids with an aliphatic tail of less than 8 carbon atoms, medium chain fatty acids with an aliphatic tail of 8 to 14 carbon atoms or long chain fatty acids with an aliphatic tail of 16 carbon atoms or more. Long chain fatty acids are preferred.

Fats rich in one or more saturated fatty acids are generally known in the art. The term “a fat rich in one or more saturated fatty acids” as used herein generally means that the weight ratio of saturated fatty acid(s) to unsaturated fatty acid(s) is at least 0.5:1, preferably at least 0.75:1. Fats rich in one or more saturated fatty acids are generally palm oil, palm kernel oil, coconut oil, cacao butter, fat of milk or milk products, lard, tallow, fat of meat or meat products. Saturated fatty acids are, for example, butanoic acid, hexanoic acid, octanoic acid (caprylic acid), decanoic acid (capric acid), dodecanoic acid (lauric acid), tetradecanoic acid (myristic acid), hexadecanoic acid (palmitic acid), octadecanoic acid (stearic acid), eicosanoic acid (arachidic acid), docosanoic acid (behenic acid), and tetracosanoic acid (lignoreric acid).

The term “a fat rich in one or more unsaturated fatty acids” as used herein generally means that the weight ratio of saturated fatty acid(s) to unsaturated fatty acid(s) is less than 0.5:1, preferably less than 0.35:1, more preferably less than 0.2:1. Fats rich in one or more unsaturated fatty acids are cottonseed oil, wheat germ oil, soy oil, olive oil, peanut oil, corn oil, sunflower oil, safflower oil, rapeseed oil, tea seed oil, canola oil, grape seed oil, sesame oil, flax seed oil, walnut oil, oils in avocados, or certain fish oils, such as oils from salmon, mackerel, herring or trout. The term “unsaturated fatty acids” as used herein means unsaturated fatty acids having a cis configuration. Unsaturated fatty acids can be monounsaturated or polyunsaturated fatty acids.

Monounsaturated fatty acids are, for example, myristoleic acid, palmitoleic acid, oleic acid and gadoleic acid. Preferred fats rich in monounsaturated fatty acids are wheat germ oil, soy oil, olive oil, peanut oil, corn oil, sunflower oil, safflower oil, rapeseed oil, tea seed oil, canola oil, sesame oil, flax seed oil, walnut oil, oils in avocados, or grape seed oil. Polyunsaturated fatty acids are, for example, omega-3 polyunsaturated fatty acids, such as alpha-linolenic acid, stearidonic acid, eicosapentaenoic acid, docosapentanoic acid, or docosahexaenoic acid; or omega-6 fatty acids, such as linoleic acid, gamma-linolenic acid, eicosadienoic acid, dihomo-gamma-linolenic acid, arachindonic acid, docosadienoic acid, adrenic acid, or docosapentaenoic acid. Preferred fats rich in omega-3 polyunsaturated fatty acids are fish oil, like oils from salmon, mackerel, herring or trout; walnut oil, rapeseed oil, soybean oil and flax seed oil. Preferred fats rich in omega-6 polyunsaturated fatty acids are sunflower seed oil, wheat germ oil, sesame oil, walnut oil, soybean oil and corn oil.

The food product of the present invention comprises at least 2 weight percent, preferably from 2 to 10 weight percent, more preferably from 2 to 6 weight percent of water-soluble cellulose derivative, based on the total weight of the food product. The daily dose of water-soluble cellulose derivative is generally in the range of 20 to 700 milligrams of water-soluble cellulose derivative per kilogram of mammal body weight per day. Preferably about 2 to 30, more preferably about 3 to 25 g of water-soluble cellulose derivative are ingested by a large mammal such as a human. The most preferred percentage of water-soluble cellulose derivative in the food product depends on various factors, such as the fat content of the diet. The water-soluble cellulose derivative is preferably incorporated in such amount in the food such that the recommended daily doses can conveniently be consumed.

The term “cellulose derivative” does not include unmodified cellulose itself which tends to be water-insoluble. The term “water-soluble” as used herein means that the cellulose derivative has a solubility in water of at least 2 grams, preferably at least 3 grams, more preferably at least 5 grams in 100 grams of distilled water at 25° C. and 1 atmosphere.

Preferred cellulose derivatives are water-soluble cellulose esters and cellulose ethers. Preferred cellulose ethers are water-soluble carboxy-C₁-C₃-alkyl celluloses, such as carboxymethyl celluloses; water-soluble carboxy-C₁-C₃-alkyl hydroxy-C₁-C₃-alkyl celluloses, such as carboxymethyl hydroxyethyl celluloses; water-soluble C₁-C₃-alkyl celluloses, such as methylcelluloses; water-soluble C₁-C₃-alkyl hydroxy-C₁₋₃-alkyl celluloses, such as hydroxyethyl methylcelluloses, hydroxypropyl methylcelluloses or ethyl hydroxyethyl celluloses; water-soluble hydroxy-C₁₋₃-alkyl celluloses, such as hydroxyethyl celluloses or hydroxypropyl celluloses; water-soluble mixed hydroxy-C₁-C₃-alkyl celluloses, such as hydroxyethyl hydroxypropyl celluloses, water-soluble mixed C₁-C₃-alkyl celluloses, such as methyl ethyl celluloses, or water-soluble alkoxy hydroxyethyl hydroxypropyl celluloses, the alkoxy group being straight-chain or branched and containing 2 to 8 carbon atoms. The more preferred cellulose ethers are methylcellulose, methyl ethyl cellulose, hydroxyethyl cellulose, hydroxypropyl cellulose, hydroxyethyl methylcellulose, hydroxypropyl methylcellulose, and carboxymethyl cellulose, which are classified as water-soluble cellulose ethers by the skilled artisans. The most preferred water-soluble cellulose ethers are methylcelluloses with a methyl molar substitution DS_{methoxyl} of from 0.5 to 3.0, preferably from 1 to 2.5, and hydroxypropyl methylcelluloses with a DS_{methoxyl} of from 0.9 to 2.2, preferably from 1.1 to 2.0, and a MS_{hydroxypropoxyl} of from 0.02 to 2.0, preferably from 0.1 to 1.2. The methoxyl content of methyl cellulose can be determined according to ASTM method D 1347-72 (reapproved 1995). The methoxyl and hydroxypropoxyl content of hydroxypropyl methylcellulose can be determined by ASTM method D-2363-79 (reapproved 1989). Methyl celluloses and hydroxypropyl methylcelluloses, such as K100M, K250M, K4M, K1M, F220M, F4M and J4M hydroxypropyl methylcellulose are commercially available from The Dow Chemical Company). Combinations of two or more water-soluble cellulose derivatives are also useful.

The water-soluble cellulose derivative generally has a viscosity of from 5 to 2,000,000 cps (=mPa·s), preferably from 50 to 1,000,000 cps, more preferably from 1,000 to 500,000 cps, in particular from 10,000 to 300,000 cps, measured as a two weight percent aqueous solution at 20 degrees Celsius. The viscosity can be measured in a rotational viscometer.

The food product of the present invention is preferably, but not limited to a processed meat product, a dairy product, a fried product or a product able to be fried, a sauce, a bakeable or baked product or a snack bar. Preferred examples of processed meat products are sausages, food products comprising chopped or sliced meat and animal fat, such as bologna, lard, salami, pate, cooked pork, pork frankfurters, or hamburgers. Preferred dairy products are milk shakes, milk shake mixes, breakfast drinks, flavored drink mixes, yogurts, puddings, ice creams, ice milks, frostings, frozen yogurts, cheesecake fillings, mayonnaise, margarine, pastry fillings, cream fillings, cheese products, and fat-containing instant potato mixes. Preferred fried products or products able to be fried are processed meat or fish products, potato products, such as potato chips or French fries, or dough products. Preferred sauces are Alfredo sauces and any other fat-containing pasta sauces, salad dressings, or other fat-containing gravies. Preferred bakeable or baked food products are cereals, bakery products such as breads, “veggie” burgers, pound cakes, doughs, pastas, cookies, biscuits, fruity snacks, muffins, or crackers. Useful bakable and baked food products can be produced as described in U.S. Pat. No. 5,281,584, provided that additionally a) a fat rich in one or more saturated fatty acids, and b) a fat rich in one or more unsaturated fatty acids is incorporated in the bakeable or baked food product. Preferred snack bars are granola bars, fruit bars or chocolate bars.

Another aspect of the present invention relates to a food product in the form of a processed meat product, a dairy product, a fried product or a product able to be fried which comprises i) one or more saturated fatty acids, ii) one or more unsaturated fatty acids, and iii) at least 2 weight percent of one or more water-soluble cellulose derivatives, based on the total weight of the food product. The saturated fatty acid(s) and the unsaturated fatty acid(s) may originate from different sources of fat or from a single source of fat. Fats, such as those listed above, typically comprise one or more saturated fatty acids and one or more unsaturated fatty acids in combination. The presence of one or more water-soluble cellulose derivatives in the food product or the administration of one or more water-soluble cellulose derivatives in combination with the food product is particularly effective for preferential excretion of saturated fatty acids relative to the excretion of unsaturated fatty acids if the food product comprises 1.5 weight percent, typically at least 2.5 weight percent, more typically at least 5 weight percent, and especially at least 10 percent of one or more saturated fatty acids and at least 1.5 weight percent, typically at least 2.5 weight percent, more typically at least 5 weight percent, and especially at least 7.5 weight percent of one or more unsaturated fatty acids. The preferred upper limit of one or more saturated fatty acids and one or more unsaturated fatty acids is mainly determined by nutricial and organoleptic considerations. Depending on the type of food, the preferred amount of total fatty acids is typically up to about 85 weight percent, of which preferably 5 to 75 weight percent originate from one or more saturated fatty acids. The weight ratio between saturated fatty acids and unsaturated fatty acids is generally from 0.05 to 15:1, preferably from 0.1 to 15:1, more preferably from 0.15 to 1.5:1, most preferably from 0.5 to 1.2:1. The given percentages relate to the total weight of saturated fatty acids, the total weight of unsaturated fatty acids and the total weight of the food product. Preferred processed meat products, dairy products, fried products and products able to be fried are described above. Preferred saturated fatty acids, unsaturated fatty acids, and water-soluble cellulose derivatives and preferred amounts of water-soluble cellulose derivatives are those described further above.

Another aspect of the present invention relates to a method of reducing the amount of saturated fatty acids capable of being absorbed by an animal body relative to the amount of unsaturated fatty acids capable of being absorbed by an animal body in or from a fat-containing food product which comprises the steps of i) identifying a food product comprising one or more saturated fatty acids and one or more unsaturated fatty acids, and ii) incorporating one or more water-soluble cellulose derivatives in the food product or administering one or more water-soluble cellulose derivatives before, during or after the consumption of the food product.

Yet another aspect of the present invention relates to a method of reducing the effective caloric content contributed by one or more saturated fatty acids relative to the caloric content contributed by one or more unsaturated fatty acids in or from a fat-containing food product comprising the steps of i) identifying a food product comprising one or more saturated fatty acids and one or more unsaturated fatty acids, and ii) incorporating one or more water-soluble cellulose derivatives in the food product or administering one or more water-soluble cellulose derivatives before, during or after the consumption of the food product.

Yet another aspect of the present invention relates to a method of reducing the amount of predominantly saturated triacylglycerides capable of being hydrolyzed in the intestine by lipases relative to the amount of predominantly unsaturated triacylglycerides capable of being hydrolyzed in the intestine by lipases

in or from a fat-containing food product comprising the steps of
i) identifying a food product comprising one or more predominantly saturated triacylglycerides and one or more predominantly unsaturated triacylglycerides, and
ii) incorporating one or more water-soluble cellulose derivatives in the food product or administering one or more water-soluble cellulose derivatives before, during or after the consumption of the food product.

The term “predominantly saturated triacylglycerides” as used herein means triacylglycerides containing no or only one C═C double bond, i.e., containing two or three esterified saturated fatty acid groups and one or zero esterified monounsaturated fatty acid group. The term “predominantly unsaturated triacylglycerides” as used herein means triacylglycerides containing two or more C═C double bonds, such as triacylglycerides containing two esterified saturated fatty acid groups and one esterified di or tri-unsaturated fatty acid group; or triacylglycerides containing zero or one esterified saturated fatty acid group and three or two esterified mono-, di- or tri-unsaturated fatty acid groups.

The term “animal” as used herein encompasses any animals including human beings. Mammals are preferred. The term “mammal” refers to any animal classified as a mammal, including human beings, domestic and farm animals, such as cows, nonhuman primates, zoo animals, sports animals, such as horses, or pet animals, such as dogs and cats. Human beings are preferred.

Surprisingly, it has been found that the preferential excretion of saturated fatty acids over unsaturated fatty acids is such that [(weight saturated fatty acids in food product) divided by (weight unsaturated fatty acids in food product)]=[(m)×(weight saturated fatty acids capable of being absorbed by animal body) divided by (weight unsaturated fatty acids capable of being absorbed by animal body)], wherein m generally is at least 1.5, typically at least 3, and many cases even at least 5, and × designates the multiplying operator.

Surprisingly, it has been found that the effective caloric content contributed by one or more saturated fatty acids generally is reduced by at least 5 percent, typically by at least 10 percent, in most cases by at least 15 percent, and in many cases even by at least 20 percent, by incorporating one or more water-soluble cellulose derivatives in the food product. In contrast thereto, it has been found that the effective caloric content contributed by one or more unsaturated fatty acids generally is reduced by less than 10 percent, typically less than 5 percent, by incorporating one or more water-soluble cellulose derivatives in the food product.

Applicants have found predominantly saturated triacylglycerides and diacylglycerides in the feces of mammals that have consumed one or more water-soluble cellulose derivates in combination with one or more fats containing predominantly saturated triacylglycerides. Without wanting to be bound by the theory, Applicants believe to have found that the hydrolysis of triacylglycerides is impaired or slowed down in an animal body by water-soluble cellulose derivates. When the rate of hydrolysis of triacylglycerides is slowed down, the rate of absorption of fatty acids by an animal body is also slowed down.

Surprisingly, it has been found that the amount of predominantly saturated triacylglycerides capable of being hydrolyzed in the intestine by lipases can be reduced to such an extent that [(weight predominantly saturated triacylglycerides in food product) divided by (weight predominantly unsaturated triacylglycerides in food product)]=[(n)×[(weight predominantly saturated triacylglycerides capable of being hydrolyzed in the intestine by lipases) divided by (weight predominantly unsaturated triacylglycerides capable of being hydrolyzed in the intestine by lipases)], wherein n generally is at least 1.5, typically at least 3, and many cases even at least 5, and × designates the multiplying operator.

The presence of one or more water-soluble cellulose derivatives in the food product or the administration of one or more water-soluble cellulose derivatives in combination with the food product is particularly effective for reducing the amount of predominantly saturated triacylglycerides capable of being hydrolyzed in the intestine by lipases if the food product comprises at least 2.5 weight percent, typically at least 5 weight percent, and more typically at least 10 percent of one or more predominantly saturated triacylglycerides and at least 2.5 weight percent, typically at least 5 weight percent, and more typically at least 7.5 weight percent of one or more predominantly unsaturated triacylglycerides. It has also been found that an optimal effect is achieved if the total fat content in the food product is at least 20 percent, more preferably at least 25 percent, based on the total weight of the food product. The preferred upper limit of one or more predominantly saturated and unsaturated triacylglycerides is mainly determined by nutricial and organoleptic considerations. Depending on the type of food, the preferred amount of total triacylglycerides is typically up to about 85 weight percent, of which preferably 5 to 75 weight percent originate from one or more predominantly saturated triacylglycerides. The percentages are by total weight of the food product. The weight ratio between predominantly saturated triacylglycerides and predominantly unsaturated triacylglycerides is generally from 0.05 to 15:1, preferably from 0.1 to 15:1, more preferably from 0.15 to 1.5:1, and most preferably from 0.5 to 1.2:1.

Preferred fat-containing food products, preferred water-soluble cellulose derivatives, preferred saturated fatty acids, preferred unsaturated fatty acids, and preferred amounts of saturated fatty acids, unsaturated fatty acids, and triacylglycerides are those described further above.

The water-soluble cellulose derivatives are preferably incorporated in such amount in the food product or are administered in such amounts before, during or after the consumption of the food product such that the daily dose of water-soluble cellulose derivative is generally in the range of 20 to 700 milligrams of water-soluble cellulose derivative per kilogram of mammal body weight per day. Preferably about 2 to 30, more preferably about 3 to 25 g of water-soluble cellulose derivative are ingested by a large mammal such as a human. The most preferred amount of water-soluble cellulose derivative depends on various factors, such as the fat content of the diet. When the water-soluble cellulose derivative is incorporated in the food product, its amount is generally at least 2 weight percent, preferably from 2 to 10 weight percent, more preferably from 2 to 6 weight percent, based on the total weight of the food product. However, in the methods of the present invention the food product can comprise less than 2 weight percent, such as 0.4 weight percent or more, preferably 1 weight percent or more, and generally up to 10 weight percent, preferably up to 6 weight percent, based on the total weight of the food product. When the water-soluble cellulose derivative is administered separately from the food product, in can, for example be administered in the form of powdered, reverse-enteric coated or micro-encapsulated cellulose ether suspended in a flavored drink formulation, as tablets, capsules, sachets or caplets. When the water-soluble cellulose derivative is administered separately from the food product, it should be consumed in an appropriate timeframe with the food product, preferably within about 30 minutes before or after consumption of the food product, more preferably within about 15 minutes before or after consumption of the food product.

The present invention is further illustrated by the following examples which are not to be construed to limit the scope of the invention. Unless otherwise indicated, all parts and percentages are by weight.

### Example 1

An animal study was conducted with male Golden Syrian hamsters with a starting body weight of between 80-90 grams (LVG strain, Charles River Laboratory, Willmington, Mass.) in each of the diets specified below. The animal study was approved by the Animal Care and Use Committee, Western Regional Research Center, USDA, Albany, Calif. The male Syrian Golden hamsters were divided into two main groups. One group was called the “treatment group” and was a fed diet comprising hydroxypropyl methylcellulose (HPMC) having a methoxyl content of 19-24 percent, a hydroxypropoxyl content of 7-12 percent and a viscosity, measured as a two weight percent aqueous solution at 20 degrees Celsius, of about 250,000 mPa·s (cps). The other group was called the “control group” and was fed a diet comprising microcrystalline cellulose (MCC). Each group consisted of approximately 10 hamsters.

Treatment Group: “Supersize (SS)” Diet, 4 weight percent HPMC

This treatment group was fed a high-fat “supersize” diet consisting of the following: 1700 g of “supersize”, 17 g of a vitamin mixture, 60 g of a mineral mixture, and 71 g of hydroxypropyl methylcellulose. “Supersize” designates meals of hamburgers/french fries that were freeze dried and powdered and that were comprised of the following energy intake: 33.62% carbohydrates, 51.75% fat, and 14.63% proteins.

Control Group: “Supersize” Diet, 4 weight percent MCC

This treatment group was fed a high-fat “supersize” diet consisting of the following: 1700 g “supersize”, 17 of a vitamin mixture, 60 g of a mineral mixture, and 71 g of microcrystalline cellulose.

All hamsters had been fed standardized laboratory food (lab “chow”) for seven days to acclimate. At the end of a 7 day period, feces were sampled for 2 days from all animals. During the feeding of the specified diet as described above feces were collected from each hamster after 10 days and 20 days. Samples were freeze dried at each collection time.

Analysis of Bile Acids, Sterols, and Acylglycerides in Feces of Hamsters Fed with “Supersize” Diet

The method for analysis of hamster fecal samples for the determination of bile acids, sterols, mono-, di-, and triacylglycerides was done by HPLC (high performance liquid chromatography). Briefly, a lyophilized, ground feces sample (0.15 g+/−0.05 g) was weighed and mixed with 3.5 g of sand in a Dionex ASE extraction cell. A 100 μL aliquot of internal standard spiking solution (500 μg/mL in tetrahydrofuran) was added to each sample (50 μg internal standard=trierucin). The cell was placed in a Dionex Accelerated Solvent Extraction (ASE) system, and the extraction was carried out with 60/40 hexane/2-propanol with 2% acetic acid at 60° C. and 2175 psi (static 10 min) The extract (20 mL) was received into a pre-weighed vial and shaken. Approximately, 9 mL of extract was transferred to a separate vial and used for gas chromatography-flame ionization detector (GC-FID) analysis for determination of fatty acid methyl esters (FAMEs), see below. Another 9 mL of extract was evaporated to dryness under a stream of nitrogen (65° C., 45 min, 8 psi). Eight mL of acetonitrile was added to the vial and it was again evaporated to dryness and constant weight under a stream of nitrogen (45° C., 45 min, 10 psi). The residue was weighed to determine percent total lipids. The residue was reconstituted in 0.9 mL of 2/6 tetrahydrofuran/[50/50 mobile phases A/B]. The solution was filtered through a 10 mm, 0.2 μm polytetrafluoroethylene syringe filter into a 2-mL HPLC autosampler vial. The sample was analyzed by HPLC using the conditions outlined below:
Instrument: Agilent 1200RR HPLC system
Column: Waters Acquity BEH C18 column, 2.1×100 mm, 1.7 μm
Column Temp.: 50° C.
Flow Rate: 0.250 mL/min
Injection Volume: 2 μL
Mobile Phase A: 53/23/24 MeOH/MeCN/H₂O with 30 mM ammonium acetate, plus 24 mL per L acetic acid
Mobile Phase B: 2-Propanol with 30 mM ammonium acetate
Gradient:

| | % Mobile |
|---|---|
| Time (min) | Phase B |
| 0.01 | 4 |
| 6.0 | 36 |
| 8.0 | 48 |
| 17.0 | 51 |
| 18.0 | 73 |
| 31.0 | 85 |
| 34.0 | 96 |
| 35.0 | 4 |

Stop time: 36.0 min; Post time: 10.0 min
Detection: ESA Biosciences, Corona Plus Charged Aerosol Detector (CAD)
Detector Nebulizer temp: 30° C.; Full scale gain range: 100 pA;
Conditions: Baseline offset: 0%; data rate: 60 pts/sec
Integration: Integration was reviewed for each file; manual splitting of merged
Events: peaks and shoulders was occasionally used to achieve appropriate, consistent reporting.

| Event | Value | Time (min) |
|---|---|---|
| Slope sensitivity | 1.50 | Initial |
| Peak width | 0.06 | Initial |
| Area reject | 0.3 | Initial |
| Height reject | 0.3 | Initial |
| Shoulders | Off | Initial |
| Integration | Off | 0.05 |
| Integration | On | 2.00 |
| Baseline Next Valley | On | 9.00 |
| Baseline Next Valley | On | 12.70 |
| Baseline Next Valley | On | 25.40 |
| Integration | Off | 34.00 |

Report: Internal Standard Report using Trierucin (RT=30.8 min) as internal standard

Each set of samples was analyzed and also included two spiked sand samples for recovery confirmation. Sand samples were spiked with lithocholic acid (LCA), cholesterol (CHOL), monopalmitin (MP), 1,3-dipalmitin (DP), and triolein (OOO) each at 50 μg and trierucin at 50 μg. Each set also included two sand/cell blank samples, spiked with 50 μg internal standard only.

In addition, a set of five calibration standards was analyzed before and after each set of samples. The calibration samples contained 25, 50, 100, 200, or 300 μg/mL each of lithocholic acid (bile acid); cholesterol (sterol); monoacylglycerides (MAG), specifically monopalmitin; diacylglycerides (DAG), specifically 1,3-dipalmitin; and triacylglycerides (TAG), specifically triolein. The set of 5 calibration standards was used to establish a 5-point internal standard linear calibration plot for each class of compounds based on the peak area of the representative class component and the peak area of the internal standard (trierucin) which was added at 50 μg level (25 μg/mL) to each standard and sample.

The correlation coefficient, R² was >0.998 for each calibration plot (area ratio vs. amount ratio).
For quantitation, peak sum windows were set up as follows:
Bile acids: 2.2-6.4 min
FFAs (free fatty acids)/MAGs: 6.4-14.1 min

### Sterols: 14.1-16.9 min

### DAGs: 17.0-25.0 min

### TAGs: 25.0-30.3 min

The reported μg/mL were converted to μg/g sample using the following equation:

$$\frac{ug}{g} = \frac{ug}{mL} \times \frac{0.9}{Wt} \times \frac{20}{9}$$

Where

μg/g=concentration of component in feces

μg/mL=concentration of component in reconstituted residue from extraction

0.9=final sample volume, mL, for HPLC analysis

Wt=initial feces weight prior to extraction (about 0.15 g)

20=extract volume, mL, (prior to split)

9=volume, mL, portion of 20 mL extract solution used for HPLC analysis (before evaporation)

### Results

Hamsters were fed a standardized laboratory food (“chow”) diet for seven days, and then changed to a celluosic-supplemented diet for 20 days. Fecal lipid levels were determined and summarized in Table 1. The data was analyzed using JMP statistical software using One Way Analysis of Variance (ANOVA) and the means tested using the Student's t-Test.

TABLE 1
Summary of average analyte class concentration for
“supersize” (SS) diet supplemented with either HPMC or MCC

| | | Average mg/g feces | | | | |
|---|---|---|---|---|---|---|
| | | Bile | | | | |
| Diet | Day | Acids | FFAs + MAGs | Sterols | DAGs | TAGs |
| Chow | 0 | 4.02 | 19.09 | 10.94 | 4.59 | 2.25 |
| SS/MCC | 10 | 11.26 | 50.84 | 33.74 | 9.70 | 4.70 |
| SS/HPMC | 10 | 25.51 | 142.68 | 26.05 | 10.12 | 4.09 |
| SS/MCC | 20 | 6.40 | 44.25 | 16.68 | 4.00 | 2.28 |
| SS/HPMC | 20 | 16.53 | 112.38 | 24.32 | 7.19 | 1.75 |

The supersize diet supplemented with HPMC showed a significant increase (p<0.05) in bile acids, free fatty acids and monoglycerides, sterols, and diacylglycerides compared to supersize diets supplemented with MCC. The analysis of fecal bile acids and sterols illustrates that water-soluble cellulose derivatives facilitate the excretion of bile acids as well as cholesterol-derived metabolites in the feces of hamsters.

### Analysis of Total Saturated Fatty Acids and Unsaturated Fatty Acids in Feces of Hamsters Fed Supersize Diet

The fecal samples were ground for 10 minutes on a Spex 8000 mixer mill using 2 tungsten carbide balls in a tungsten carbide cylinder. Samples were ground for 10 minutes. An aliquot of about 0.35 g diatomaceous earth (sand) was added to a Dionex 11 mL Accelerated Solvent Extractor (ASE) cell. An aliquot (0.15 g) of ground fecal matter was weighed into the cell and the fecal sample and sand mixture were briefly stirred. The cells were then spiked with 100 μL of a 500 μg/mL solution of glyceryl trierucate in tetrahydrofuran (THF). The mixture was sandwiched between two cellulose filters. Extraction solvent contained 600 mL of hexane, 400 mL 2-propanol and 20 mL acetic acid. The cell contents were extracted on a Dionex ASE system with the following conditions: Preheat 1 min, pressure 2175 psi, heat 5 min, Temp 60° C., Static 10 min, flush 60%, purge 120 sec, cycle=2. This resulted in 20 mL of sample extract collected.

After mixing, 9.0 mL of extract was placed in a tared 16×125 mm screw top culture tube. The extract was blown to dryness in a 45° C. water bath with a nitrogen purge. A 4 mL aliquot of acetonitrile was added and the mixture was blown to dryness again. The sample was brought to constant weight in a dessicator and weighed to gravimetrically determine total extractables (“total lipids”).

The samples were derivatized using the following procedure:
1. A 300 μL aliquot of 0.5N NaOH in MeOH (methanol) was added to each sample in the culture tube.
2. Tubes were capped, vortexed, and placed in a heating block at 100 C for 5 min
3. Samples were allowed to cool about 1 min
4. Samples were uncapped and 350 μL of 14% BF₃ in MeOH was added.
5. Samples were capped, vortexed, and placed in a heating block at 100° C. for 5 min
6. Samples were allowed to cool about 1 min
7. Vials were uncapped and 2 mL heptane (0.200 mg/mL nonadecane in heptane) was added.
8. Vials were recapped, vortexed, and placed back in heating block at 100 C for 5 min.
9. Samples were allowed to cool for about 1 min
10. Vials were uncapped and 1 mL NaCl saturated H₂O solution was added.
11. They were recapped and placed on rocker for 5 min
12. They were then centrifuged at 1500 rpm for 10 min
13. Using Pasteur pipettes, for about 1 mL of organic (top) layer was transferred into gas chromatography (GC) vials.

The derivatized samples were analyzed by gas chromatography with the following conditions:

| | |
|---|---|
| Chromatograph: | Agilent 6890 Series GC |
| Column: | 60 m × 0.25 mm (L × ID), 0.25 μm df, |
| Detector | FID (Flame Ionization Detector) |
| Temperatures: | |
| Oven: | 200° C. (5 min), about 250° C. at 5° C./min |
| | with 5 min. final hold time |
| Injector: | 250° C. |
| Detector: | 260° C. |
| Carrier: | 3 mL/min at 200° C. (62.5 psi) |
| Split: | 25 mL/min |
| Make-Up: | Helium 27 mL/min |
| Air: | 400 mL/min |
| Hydrogen: | 30 mL/min |
| Sample Size: | 2 μL |
| Data System: | EZChrom Elite Version 3.2.1 |

For calibration, a spiking standard was prepared to contain 0.200 mg/mL nonadecane and 0.10 mg/mL methyl erucate in heptane. A calibration solution was prepared by weighing out 10 mg of NuCheck Standard 1A into a 1.0 mL volumetric flask. To this flask 110 μL of the spiking standard was added. The flask was diluted to volume with heptane containing 0.200 mg/mL nonadecane. NuCheck Standard 1A contained 20% each of methyl palmitate (C16:0), methyl stearate (C18:0), methyl oleate (C18:1), methyl linoleate (C18:2) and methyl linolenate (C18:3). Thus the calibration solution contained 2000 μg/mL of these five components along with 200 μg/mL nonadecane and 11 μg/mL methyl erucate.

Total saturated fatty acids were the sum of C14:0 through C22:0. Total unsaturated fatty acids were the sum of monounsaturated fatty acids C14:1 through C20:1 plus C18:2 and C18:3. C18:2 and C18:3 account for more than 90% of all polyunsaturated fatty acids.

### Results

To further illustrate the selectivity of hydroxypropyl methylcellulose on lipid levels, the ratio of saturated fatty acids/unsaturated fatty acids (SATs/UNSATs) was determined by comparing the starting food composition to the feces. The data was analyzed using JMP statistical software. Within each group the levels of species of interest were analyzed with JMP using Means Anova Pooled t-Test. The food composition and the feces analysis of the summation of saturated and unsaturated fatty acids are presented in Table 2.

TABLE 2
Summary of total saturated and unsaturated fatty acids from “supersize”
food composition and feces collected at day 10 and day 20 from hamsters
fed “supersize” diet with HPMC

| | FAME | FAME | | |
|---|---|---|---|---|
| | SATs | UNSATs | | Total Fatty |
| | Sum mg/g | Sum mg/g | | Acids [SATs + |
| | AVG* all | AVG* all | SATs/UNSATs | UNSATs] |
| Day/Additive | animals | animals | Ratio | mg/g |
| Food | 73 | 147 | 0.5 | 220 |
| Composition | | | | |
| Day 10 Feces, | 164 | 50 | 3.3 | 214 |
| HPMC | | | | |
| Day 20 Feces, | 120 | 33 | 3.7 | 153 |
| HPMC | | | | |

*Average

Hamsters fed the “supersize” diet supplemented with 4% hydroxypropyl methylcellulose had a significantly higher SATs/UNSATs ratio in the feces compared to the SATs/UNSATs ratio in the starting “supersize” food composition at both day 10 and day 20. Thus, hydroxypropyl methylcellulose significantly altered the ratio of total saturated fatty acids relative to unsaturated fatty acids when comparing the SATs/UNSATs ratio of the starting food composition relative to the excreted feces. The HPMC facilitated the preferential excretion of the saturated fatty acids from hamsters fed a high-fat “supersize” meal. Table 2 illustrates that water-soluble cellulose derivatives are useful for reducing the amount of saturated fatty acids capable of being absorbed by an animal body relative to the amount of unsaturated fatty acids capable of being absorbed by an animal body after the consumption of a fat-containing food product.

### Analysis of Triacylglycerides in Hamster Feces

### Extraction of Feces

A sample of dry feces (0.1 g) was transferred to a sample vial, to which the internal standards were added: 100 μl of triheptadecenoin and 100 μl of trinonadecenoin dissolved in 500 mg/ml dissolved in a mixture of methanol and isopropyl alcohol. The sample vial was loaded on a Dionex ASE 200 automatic extractor (Dionex Corp., Sunnyvale, Calif.). Extraction was performed using a 20 mL mixture of hexane and isopropyl alcohol (3:2, v/v, 2% acetic acid) at 2178 psi, 60° C. for 30 min. After extraction, the solvent was evaporated under N₂ stream, and the extract was reconstituted in 400 μL isopropyl alcohol, then filtered using a 0.45-mm polytetrafluoroethylene syringe membrane filter, and 40 μL was injected onto the HPLC.

### Analytical Procedure:

The fecal extracts obtained from the fat extractor were analyzed on an LC system (Surveyor system including LC pump, autosampler, photo diode array detector, Thermo Finnigan, San Jose, Calif.) using a Luna C18 column (3 μm, 150×2.0 mm; Phenomenex, Torrance, Calif.) that were compatible with the mass spectrometer (MS). The outlet of the PDA detector of the liquid chromatograph was connected to a Finnigan LCQ™ quadrupole ion trap mass spectrometer (Thermo Finnigan, Inc. Waltham, Mass.) through an APCI (Atmospheric Pressure Collision Induced) source in positive ion mode. Data was collected and processed using Excalibur software (Ver. 1.3, Thermo Finnigan, San Jose, Calif.).

LC separation: Separation was performed using a gradient of two mobile phases: (A) methanol/acetonitrile/water (53:23:24, v/v) and (B) isopropyl alcohol (100%). To both phases an appropriate amount of crystalline ammonium acetate was added and mixed to form a 30 mM solution, followed by the addition of 20-30 mL of glacial acetic acid. A linear gradient from A to B at a flow rate of 0.1 mL/min was performed as follows: time 0-30 min, 8-36% B; time 30-40 min, 36-50% B; time 40-100 min, 50-56% B; 100-105 min, 56-70% B; 105-145 min, 70-88% B; and 145-170 min, 88-95.5% B. In all experiments, the columns were re-equilibrated between injections with the equivalent of 10 mL of the initial mobile phase.

APCI-MS condition: The optimized operating parameters of the APCI-MS interface were as follows: vaporization temperature 400° C.; capillary temperature 280° C.; capillary voltage 10 V; corona discharge 5 μA; sheath N₂ gas flow 80 arbitrary (instrument) unit; aux gas flow 0 arbitrary unit; tube lens offset 10 V; and ion collection time 200 ms. Each run was scanned at the m/z (mass to charge) values corresponding to the ammonium adduct ions of triacylglyceride ([M+NH₄]⁺) using selective ion monitoring scan mode. Table 3 summarizes the abbreviations of fatty acids. Table 4 shows all the triacylglycerides that were monitored for both diets and feces with the m/z values of ions formed from respective triacylglycerides.

TABLE 3
The abbreviation of fatty acids

| Fatty acids | No of carbon atoms | No of C═C double bonds |
|---|---|---|
| Cy: Caprylic acid | 8 | 0 |
| C: Capric acid | 10 | 0 |
| La: Lauric acid | 12 | 0 |
| M: Myristic acid | 14 | 0 |
| P: Palmitic acid | 16 | 0 |
| Po: Palmitoleic acid | 16 | 1 |
| Ma: Margaric acid | 17 | 0 |
| S: Stearic acid | 18 | 0 |
| O: Oleic acid | 18 | 1 |
| L: Linoleic acid | 18 | 2 |
| Ln: Linolenic acid | 18 | 3 |
| A: Arachidonic acid | 20 | 0 |
| G: Gadoleic acid | 20 | 1 |
| B: Behenic acid | 22 | 0 |
| Lg: Lignoceric acid | 24 | 0 |

TABLE 4
The triacylglycerides (TAG) in diets and hamsters identified
using LC/APCI-MS.

| ECN | m/z (+) | TAG |
|---|---|---|
| 24 | 566 | CyCyCy |
| 26 | 594 | CyCyC |
| 28 | 544 | CyCC |
| | 622 | CyCyLa |
| 30 | 572 | CCC |
| | 598 | CyPoCy |
| | 624 | CyCyL |
| | 650 | CyCLa |
| 32 | 600 | LaCC, CyCyP |
| | 626 | CyPoC, CyOCy |
| | 652 | CLCy |
| | 678 | CyLaLa |
| 34 | 628 | CLaLa, CCyP |
| | 654 | CPoC, LaPoCy, COCy |
| | 680 | CLC, CyLLa |
| | 706 | CLnLa |
| 36 | 656 | LaLaLa |
| | 682 | LaPoC, MPoCy, LaOCy, |
| | | COC |
| | 708 | PoPoCy, CLLa, CyLM |
| | 734 | LaLnLa |
| | 890 | LnLnLn, OOMa |
| 38 | 684 | MLaLa |
| | 710 | LaOC, MOCy |
| | 736 | PoOCy, LaLLa, CLM |
| | 892 | LLnLn, SOMa |
| 40 | 816 | LLLa |
| | 712 | PLaLa, MMLa |
| | 738 | LaOLa, POCy |
| | 764 | OOCy, MLLa |
| | 842 | PoLnPo |
| | 868 | LnLnP |
| | 894 | LLLn, OLnLn |
| 42 | 740 | PMLa |
| | 766 | MOLa, SOCy |
| | 792 | PoPoM, OOC, MLM, |
| | | PLLa |
| | 818 | PoPoPo, PoLM, OLLa |
| | 844 | PoLPo, LLM |
| | 870 | LLPo, LnLP |
| | 896 | OLLn, SLnLn, LLL |
| 44 | 768 | SMO |
| | 794 | PPoM |
| | 820 | OPoM, PPoPo, PLM, SLLa, OOLa |
| | 846 | OLM, OPoM, PPoPo, PLM, SLLa, |
| | | OOLa |
| | 872 | OLPo, LLP, LnOP |
| | 898 | OLL, OLnO, SLLn |
| | 924 | ALnLn |
| 46 | 796 | PPM |
| | 822 | POM, PPoP, SOLa |
| | 848 | OOM, POPo, PLP, SLM, GOLa |
| | 874 | LOP, SLnP, OOPo, OLP |
| | 900 | OLO, SLL, SOLn |
| | 926 | GLL, ALLn |
| | 952 | BLnLn |
| 48 | 824 | PPP |
| | 850 | POP |
| | 876 | OOP, SLP, OOO, GOM |
| | 902 | SLO, OOO |
| | 928 | ALL |
| | 854 | BLLn |
| 50 | 852 | SPP |
| | 878 | SOP, AOM, BOLa |
| | 904 | SOO, SLS, GOP, ALP |
| | 930 | GOO, GLS, ALO |
| 52 | 906 | SOS, AOP, LgOLg |
| | 880 | SSP |
| | 932 | GOS, AOO, LgLM, BLP, ALS |
| | 958 | BLO |
| | 984 | LgLL |
| 54 | 908 | SSS |
| | 934 | AOS |
| | 960 | BLS, LgLP, BOO, LgLO |
| | 986 | LgLO |
| 56 | 936 | AAP, ASS |
| | 962 | BOS, LgOP, AAO |
| | 988 | LgLS, LgOO |
| 58 | 964 | AAS |
| | 990 | ABPo, ALgM, ABO |
| 60 | 992 | AAA |
| 62 | 1120 | AAB |
| 64 | 1148 | ABB |
| 66 | 1176 | BBB |

### Calculation:

Abbreviation for triacylglyceride classification based upon “Degree of Saturation”

### 0: S₀

### 1: S₁

### 2: S₂

### 3: S₃

### 0 or 1: S₀₁

### 1 or 2: S₁₂

### 2 or 3: S₂₃

### Where:

S₀=triacylglyceride with at least three C═C double bonds
S₁=triacylglyceride with two C═C double bonds
S₂=triacylglyceride with one C═C double bond
S₃=triacylglyceride with no C═C double bond
S₀₁=S₀ or S₁ in cases where S₀ and S₁ cannot be distinguished by combined MS/HPLC
S₁₂=S₁ or S₂ in cases where S₁ and S₂ cannot be distinguished by combined MS/HPLC
S₂₃=S₂ or S₃ in cases where S₂ and S₃ cannot be distinguished by combined MS/HPLC
Predominantly saturated is a triacylglyceride with 0 or only 1 C═C double bond.
Predominantly unsaturated is a triacylglyceride with two or more C═C double bonds.
The degree of saturation is determined by:

Ratio=(S₂+S₃+S₂₃)/(S₀+S₁+S₀₁+S₁₂).

### Results

The fatty acid profiles of the excreted lipids were analyzed. The focus was on the triacylglyceride fractions. Since the extracted lipids were initially separated and their lipid classes analyzed by HPLC, the same methodology coupled with mass spectrometry (MS) was chosen to assist in identifying the triacylglycerides. Triacylglycerides are separated by effective carbon number (ECN). ECN is defined as the number of carbons contributed by the fatty acid, CN (carbon number), less two carbons for every double bond (DB) or ECN=CN−2×DB. Thus, triolein, 3×18:1 would have a ECN of 3×18−2×3 or 48. Tripalmitin would have the same ECN. These would not be separable by HPLC but would be separated by selective mass monitoring. However, triolein would not be distinguishable from SOLn or 18:0, 18:1, 18:2 by HPLC/MS since the ECN and mass would be the same. The primary objective was to determine the degree of saturation for each triacylglyceride by the combination of ECN and mass (see Table 5).

TABLE 5
Triacylglyceride classification based upon the degree of saturation.

| | m/z | | | Degree |
|---|---|---|---|---|
| ECN | (+) | RT | TAG | of SAT |
| 34 | 654 | 15.01 | CPoC, LaPoCy, COCy | 2 |
| | 924 | 49.42 | ALnLn | 1 |
| | 706 | 16.26 | CLnLa | 2 |
| 36 | 656 | 17.12 | LaLaLa | 3 |
| | 682 | 17.82 | LaPoC, MPoCy, LaOCy, COC | 2 |
| | 708 | 18.86 | PoPoCy, CLLa, CyLM | 1 or 2 |
| | 734 | 19.37 | LaLnLa | 2 |
| | 890 | 24.04 | LnLnLn | 0 |
| 38 | 684 | 20.88 | MLaLa | 3 |
| | 710 | 21.71 | LaOC, MOCy | 2 |
| | 736 | 22.39 | PoOCy, LaLLa, CLM | 1 or 2 |
| | 892 | 23.7 | LLnLn | 0 |
| 40 | 712 | 24.87 | PLaLa, MMLa | 3 |
| | 738 | 26.12 | LaOLa, POCy | 2 |
| | 764 | 26.87 | OOCy, MLLa | 1 or 2 |
| | 842 | 32.25 | PoLnPo | 0 |
| | 868 | 34.15 | LnLnP | 1 |
| | 894 | 33.26 | LLLn, OLnLn | 0 |
| 42 | 740 | 30.22 | PMLa | 3 |
| | 766 | 31.14 | MOLa, SOCy | 2 |
| | 792 | 32.36 | PoPoM, OOC, MLM, PLLa | 1 or 2 |
| | 818 | 33.85 | PoPoPo, PoLM, OLLa | 0 or 1 |
| | 844 | 35.36 | PoLPo, LLM | 0 or 1 |
| | 870 | 36.92 | LLPo, LnLP | 0 or 1 |
| | 896 | 37.86 | OLLn, SLnLn, LLL | 0 or 1 |
| 44 | 768 | 36.46 | SMO | 2 |
| | 794 | 37.86 | PPoM | 2 |
| | 820 | 39.26 | OPoM, PPoPo, PLM, SLLa, OOLa | 1 or 2 |
| | 846 | 40.65 | OLM, OPoM, PPoPo, SLLa, OOLa | 1 or 2 |
| | 924 | 49.42 | ALnLn | 1 |
| 46 | 796 | 44.57 | PPM | 3 |
| | 822 | 46.38 | POM, PPoP, SOLa | 2 |
| | 848 | 47.89 | OOM, POPo, PLP, SLM, GOLa | 1 or 2 |
| | 874 | 49.59 | LOP, SLnP, OOPo, OLP | 1 or 2 |
| | 900 | 51.82 | OLO, SLL, SOLn | 0 or 1 |
| | 926 | 54.2 | GLL, ALLn | 0 or 1 |
| 48 | 824 | 55.22 | PPP | 3 |
| | 850 | 56.42 | POP | 2 |
| | 876 | 58.52 | OOP, SLP, OOO, GOM | 0 or 1 |
| | 902 | 61.44 | SLO, OOO | 0 or 1 |
| | 928 | 63.93 | ALL | 1 |
| 50 | 852 | 67.61 | SPP | 3 |
| | 878 | 70 | SOP, AOM, BOLa | 2 |
| | 904 | 72.71 | SOO, SLS, GOP, ALP | 1 or 2 |
| | 930 | 75.06 | GOO, GLS, ALO | 0 or 1 |
| 52 | 906 | 86.53 | SOS, AOP, LgOLg | 2 |
| | 880 | 83.76 | SSP | 3 |
| | 932 | 89.1 | GOS, AOO, LgLM, BLP, ALS | 1 or 2 |
| | 958 | 93.61 | BLO | 1 |
| | 984 | 99.92 | LgLL | 1 |
| 54 | 908 | 103.78 | SSS | 3 |
| | 934 | 105.84 | AOS | 2 |
| | 960 | 108.27 | BLS, LgLP, BOO. LgLO | 1 or 2 |
| | 986 | 111.81 | LgLO | 1 |
| 56 | 936 | 116.78 | AAP, ASS | 3 |
| | 962 | 118.54 | BOS, LgOP, AAO | 2 |
| | 988 | 120.66 | LgLS, LgOO | 1 or 2 |
| 58 | 964 | 130.08 | AAS | 3 |
| | 990 | 131.86 | ABPo, ALgM, ABO | 2 or 3 |

Where:
TAG = triacylglycerides;
RT = retention time;
SAT = degree of saturation;
m/z = mass to charge; and
ECN = effective carbon number.

In this study, the data showed that the triacylglyceride fraction containing one double C═C bond is dominant Triacylglycerides containing only one double bond are predominantly saturated since they contain two saturated fatty acids. Thus, the fully saturated triacylglycerides and the triacylglycerides containing only one double bond were combined in a predominantly saturated group. Triacylglycerides containing two or more double bonds were combined in a predominantly unsaturated group. The ratios of the groups predominantly saturated triacylglycerides, SATs, to predominantly unsaturated triacylglycerides, UNSAT, were compared.

The SATs/UNSATs ratio of the triacylglycerides of the feces from the “supersize” fed group was significantly increased (p<0.1) compared to the control diet. The SATs/UNSATs ratio in the “supersize” diet of the treatment group and of the control group was 0.5 or less. The SATs/UNSATs ratio of the triacylglycerides in the feces of the treatment group fed “supersize” diet comprising 4 weight percent HPMC was 0.59, whereas the SATs/UNSATs ratio of the triacylglycerides in the feces of the control group fed “supersize” diet comprising 4 weight percent MCC was only 0.29. Thus, hydroxypropyl methylcellulose increase preferential excretion of saturated triacylglycerides in diets consisting of “supersize” meal compared with the same diets fed with microcrystalline cellulose. In addition, this unexpected observation was correlated to diets having a total fat percent greater than 20%. The amount of fat excreted by hydroxypropyl methylcellulose was about 100% greater than with MCC feeding. Just as important as the amount of saturated triacylglycerides unavailable for hydrolysis and thus reduced absorbability of free saturated fatty acids is the conclusion that absorption of saturated fatty acids is slowed down or impaired significantly by the presence of HPMC.

Collectively, this study shows that water-soluble cellulose derivatives, such as hydroxypropyl methylcellulose, preferentially interact with saturated triacylglcyerides in a high fat diet.

### Example 2

An animal study was conducted with male Golden Syrian hamsters with a starting body weight of between 80-90 grams (LVG strain, Charles River Laboratory, Willmington, Mass.) in each of the diets specified below. The animal study was approved by the Animal Care and Use Committee, Western Regional Research Center, USDA, Albany, Calif. The male Syrian Golden hamsters were divided into two main groups. One group was called the “treatment group” and was fed a diet comprising hydroxypropyl methylcellulose (HPMC), a water-soluble cellulose ether. The other group was called the “control group” and was fed a diet comprising microcrystalline cellulose (MCC). Each group consisted of approximately 10 hamsters.

Treatment Group: “Potato Chip” Diet, 4 weight percent HPMC. This treatment group was fed a high-fat “potato chip” diet consisting of the following: 56 g potato chips (freeze dried and powdered), 12 g of casein, 0.3 g of DL-methionine, 20.4 g of cornstarch, 2.5 g of corn oil, 0.3 g of choline biturate, 1 g of a vitamin mixture, 3.5 g of a mineral mixture, and 4 g of the same hydroxypropyl methylcellulose as in Example 1.

Control Group: “Potato Chip” Diet, 4 weight percent MCC

This treatment group was fed a high-fat “potato chip” diet consisting of the following: 56 g potato chips (freeze dried and powdered), 12 g of casein, 0.3 g of DL-methionine, 20.4 g of cornstarch, 2.5 g of corn oil, 0.3 g of choline biturate, 1 g of a vitamin mixture, 3.5 g of a mineral mixture, and 4 g of microcrystalline cellulose.

All hamsters had been fed standardized laboratory food (lab chow) for seven days to acclimate. At the end of a 7 day period, feces were sampled for 2 days from all animals. During the feeding of the specified diet as described above feces were collected from each hamster after 6 days. Samples were freeze dried at each collection time.

### Analysis of Bile Acids, Sterols, and Acylglycerides in Hamster Feces

The method for analysis of hamster fecal samples for the determination of bile acids, sterols, mono-, di-, and tri-acylglycerides was done by HPLC as previously described in Example 1.

### Results

Hamsters were fed a chow diet for seven days, and then changed to a celluosic-supplemented diet (potato chips) for 6 days. Fecal lipid levels were determined and summarized in Table 6. The data was analyzed using JMP statistical software using One Way Analysis of Variance (ANOVA) and the means tested using the Student's t-Test.

TABLE 6
Summary of average analyte class concentration for potato
chip (CHP) diet supplemented with either HPMC or MCC

| | | Average mg/g feces | | | | |
|---|---|---|---|---|---|---|
| | | Bile | | | | |
| Diet | Day | Acids | FFAs/MAGs | Sterols | DAGs | TAGs |
| Chow | 0 | 4.02 | 19.09 | 10.94 | 4.59 | 2.25 |
| CHP/MCC | 6 | 2.68 | 35.33 | 9.97 | 5.40 | 1.01 |
| CHP/HPMC | 6 | 19.22 | 114.45 | 15.16 | 5.88 | 1.67 |

The potato chip diet supplemented with HPMC showed a significant increase (p<0.05) in bile acids, free fatty acids/monoglycerides (FFAs/MAGs), and sterols compared to the potato chip diet supplemented with MCC. While not statistically significant (p<0.05) the potato chip diet supplemented with HPMC showed an increase in both diacylglycerides and triacylglycerides compared to the potato chip diet supplemented with MCC. Table 6 illustrates that water-soluble cellulose derivatives are useful for preferentially reducing the amount of saturated fatty acids capable of being absorbed by an animal body relative to the amount of unsaturated fatty acids capable of being absorbed by an animal body after the consumption of a fat-containing food product. The analysis of fecal bile acids and sterols illustrates that water-soluble cellulose derivatives, such as hydroxypropyl methylcellulose facilitate the excretion of bile acids as well as cholesterol-derived metabolites in the feces of hamsters.

### Analysis of Total Saturated Fatty Acids and Unsaturate Fatty Acids in Hamster Feces

The method for analysis of hamster fecal samples for the determination of total saturated fatty acids and total unsaturated fatty acids were done by FAME (Fatty Acid Methyl Ester) analysis as previously described in Example 1.

### Results

To further illustrate the selectivity of hydroxypropyl methylcellulose on lipid levels, the ratio of saturated fatty acids/unsaturated fatty acids (SATs/UNSATs) was determined by comparing the starting food composition to the feces. The data was analyzed using JMP statistical software. Within each group the levels of species of interest were analyzed with JMP using Means Anova Pooled t-Test. The food composition and the feces analysis of the summation of saturated and unsaturated fatty acids are presented in Table 7.

TABLE 7
Summary of total saturated and unsaturated fatty acids from
potato chip diet and feces collected at day 6

| | | FAME | | Total |
|---|---|---|---|---|
| | FAME SATs | UNSATs | | Fatty Acids |
| | Sum mg/g | Sum mg/g | | [SATs + |
| | AVG* all | AVG* all | SATs/UNSATs | UNSATs] |
| Day/Additive | animals | animals | Ratio | mg/g |
| Food | 81 | 147 | 0.5 | 228 |
| Composition | | | | |
| Day 6 Feces, | 102 | 29 | 3.5 | 131 |
| HPMC | | | | |

*Average

Hamsters fed the potato chip diet supplemented with 4% hydroxypropyl methylcellulose had significantly higher SATs/UNSATs ratio in the feces compared to the SATs/UNSATs ratio in the starting potato chip diet. Thus, hydroxypropyl methylcellulose significantly altered the ratio of total saturated fatty acids relative to unsaturated fatty acids when comparing the SATs/UNSATs ratio of the starting food composition relative to the excreted feces. These observations correlate with the observations made for bile acids, sterols, free fatty acids, monoacylglycerides, diacylglycerides, and triacylglycerides. Overall the HPMC facilitated the excretion of the saturated fatty acids from hamsters fed a high-fat potato chip diet. Table 7 illustrates that water-soluble cellulose derivatives are useful for reducing the amount of saturated fatty acids capable of being absorbed by an animal body relative to the amount of unsaturated fatty acids capable of being absorbed by an animal body after the consumption of a fat-containing food product.

### Analysis of Triacylglycerides in Hamster Feces Fed Potato Chip Diet

In order to determine if a water-soluble cellulose derivative, such as hydroxypropyl methylcellulose, increased the selectivity of the fatty acid profile of the excreted lipids, hamster feces were analyzed by HPLC coupled with a mass spectrometry as described in Example 1. Again triacylglycerides were separated by effective carbon number (ECN). The primary objective was to determine the degree of saturation for each triacylglyceride by the combination of ECN and mass (see Table 5).

In this study, the data showed that the triacylglyceride fraction containing one single bond was dominant. Predominantly saturated triacylglycerides and predominantly unsaturated triacylglycerides were combined in groups as described in Example 1. The ratios of the groups predominantly saturated triacylglycerides, SATs, to predominantly unsaturated triacylglycerides, UNSATs were compared.

The SATs/UNSATs ratio in the potato chip diet of the treatment group and of the control group was 0.5 or less. The SATs/UNSATs ratio of the triacylglycerides in the feces of the treatment group fed potato chip diet comprising 4 weight percent HPMC was 0.90, whereas the SATs/UNSATs ratio of the triacylglycerides in the feces of the control group fed potato chip diet comprising 4 weight percent MCC was only 0.11. The SATs/UNSATs ratio of the triacylglycerides of the feces from the potato chip fed treatment group was significantly increased (p<0.05) compared to the group fed the control diet. Thus, the presence of hydroxypropyl methylcellulose in the potato chip diet caused preferential excretion of saturated triacylglycerides, as compared with a comparable diet comprising microcrystalline cellulose. In addition, this unexpected observation was correlated to a diet having a total fat percent greater than 20%. The amount of fat excreted by hydroxypropyl methylcellulose was about 100% greater than with MCC feeding. Just as important as the amount of saturated triacylglycerides unavailable for hydrolysis and thus reduced absorbability of free saturated fatty acids is the conclusion that absorption of saturated fatty acids is slowed down or impaired significantly by the presence of HPMC.

Collectively, this study shows that water-soluble cellulose derivatives, such as hydroxypropyl methylcellulose, preferentially interact with saturated triacylglcyerides in a high fat diet.

### Example 3

An animal study was conducted with male Golden Syrian hamsters with a starting body weight of between 80-90 grams (LVG strain, Charles River Laboratory, Willmington, Mass.) in each of the diets specified below. The animal study was approved by the Animal Care and Use Committee, Western Regional Research Center, USDA, Albany, Calif. The male Syrian Golden hamsters were divided into two main groups. One group was called the “treatment group” and was fed a diet comprising hydroxypropyl methylcellulose (HPMC), a water-soluble cellulose ether. The other group was called the “control group” and was fed a diet comprising microcrystalline cellulose (MCC). Each group consisted of approximately 10 hamsters.

Treatment Group: “Cheese” Diet, 4 weight percent HPMC

This treatment group was fed a “cheese” diet consisting of the following: 35 g cheese (freeze dried and powdered), 8 g of casein, 0.3 g of DL-methionine, 45.4 g of cornstarch, 2.5 g of corn oil, 0.3 g of choline biturate, 1 g of a vitamin mixture, 3.5 g of a mineral mixture, and 4 g of the same hydroxypropyl methylcellulose as in Example 1.

Control Group: “Cheese” Diet, 4 weight percent MCC

This treatment group was fed a “cheese” diet consisting of the following: 35 g cheese (freeze dried and powdered), 8 g of casein, 0.3 g of DL-methionine, 45.4 g of cornstarch, 2.5 g of corn oil, 0.3 g of choline biturate, 1 g of a vitamin mixture, 3.5 g of a mineral mixture, and 4 g of microcrystalline cellulose.

All hamsters had been fed laboratory standard diet (lab “chow”) for seven days to acclimate. At the end of a 7 day period, feces were sampled for 2 days from all animals During the feeding of the specified diet as described above feces were collected from each hamster after 6 days. Samples were freeze dried at each collection time.

### Analysis of Bile Acids, Sterols, and Acylglycerides in Hamster Feces Fed Cheese

The method for analysis of hamster fecal samples for the determination of bile acids, sterols, mono-, di-, and tri-acylglycerides was done by HPLC as previously described in Example 1.

### Results

Hamsters were fed a chow diet for seven days, and then changed to a celluosic-supplemented diet (cheese) for 6 days. Fecal lipid levels were determined and summarized in Table 8. The data was analyzed using JMP statistical software using One Way Analysis of Variance (ANOVA) and the means tested using the Student's t-Test.

TABLE 8
Summary of average analyte class concentration
for cheese (CHS) diet supplemented with either HPMC or MCC.

| | | Average mg/g feces | | | | |
|---|---|---|---|---|---|---|
| | | Bile | | | | |
| Diet | Day | Acids | FFAs/MAGs | Sterols | DAGs | TAGs |
| Chow | 0 | 4.02 | 19.09 | 10.94 | 4.59 | 2.25 |
| CHS/MCC | 6 | 2.90 | 68.89 | 16.36 | 5.87 | 0.99 |
| CHS/HPMC | 6 | 6.33 | 119.22 | 20.53 | 7.02 | 1.26 |

The cheese diet supplemented with HPMC showed a significant increase (p<0.05) in bile acids, free fatty acids/monoglycerides (FFAs/MAGs), and sterols compared to cheese diet supplemented with MCC, control diets. While not statistically significant (p<0.05), the cheese diet supplemented with HPMC showed an increase in both diacylglycerides and triacylglycerides compared to the cheese diet supplemented with MCC. Table 8 illustrates that water-soluble cellulose derivatives are useful for reducing the amount of saturated fatty acids capable of being absorbed by an animal body relative to the amount of unsaturated fatty acids capable of being absorbed by an animal body after the consumption of a fat-containing food product. The analysis of fecal bile acids and sterols illustrates that water-soluble cellulose derivatives, such as hydroxypropyl methylcellulose, facilitate the excretion of bile acids as well as cholesterol-derived metabolites in the feces of hamsters.

### Analysis of Total Saturated and Unsaturated Fatty Acids in Feces of Hamsters Fed Cheese

The method for analysis of hamster fecal samples for the determination of total saturated and total unsaturated fatty acids were done by FAME (Fatty Acid Methyl Ester) analysis as previously described in Example 1.

### Results

To further illustrate the selectivity of hydroxypropyl methylcellulose on lipid levels, the ratio of saturated fatty acids/unsaturated fatty acids (SATs/UNSATs) was determined by comparing the starting food composition to the feces. The data was analyzed using JMP statistical software. Within each group the levels of species of interest were analyzed with JMP using Means ANOVA Pooled t-Test. The food composition and the feces analysis of the summation of saturated and unsaturated fatty acids are presented in Table 9.

TABLE 9
Summary of total saturated and unsaturated fatty acids from cheese
diet and feces collected at day 6 from hamsters fed the cheese diet

| | Fecal | Fecal FAME | | Total |
|---|---|---|---|---|
| | FAME SATs | UNSATs | | Fatty Acids |
| | Sum mg/g | Sum mg/g | | [SATs + |
| Day/ | AVG* all | AVG* all | SATs/UNSATs | UNSATs] |
| Additive | animals | animals | Ratio | mg/g |
| Food | 85 | 65 | 1.3 | 150 |
| Composition | | | | |
| Day 6 Feces, | 138 | 22 | 6.3 | 160 |
| HPMC | | | | |

*Average

Hamsters fed the cheese diet supplemented with 4% hydroxypropyl methylcellulose had a significantly higher SATs/UNSATs ratio in the feces compared to the SATs/UNSATs ratio in the starting cheese diet. Thus, hydroxypropyl methylcellulose significantly altered the ratio of total saturated fatty acids relative to unsaturated fatty acids when comparing the SATs/UNSATs ratios of the starting cheese diet relative to the excreted feces. These observations correlate with the bile acids, sterols, free fatty acids, mono acylglycerides, diacylglycerides, and triacylglycerides. Overall the HPMC facilitated the excretion of the saturated fatty acids from hamsters fed a cheese diet. Table 9 illustrates that water-soluble cellulose derivatives are useful for reducing the amount of saturated fatty acids capable of being absorbed by an animal body relative to the amount of unsaturated fatty acids capable of being absorbed by an animal body after the consumption of a fat-containing food product.

Analysis of Triacylglycerides in Feces from Hamsters Fed a Cheese Diet

In order to determine if a water-soluble cellulose derivative, such as hydroxypropyl methylcellulose, increased the selectivity of the fatty acid profile of the excreted lipids, hamster feces were analyzed by HPLC coupled with a mass spectrometry as described in Example 1. Again triacylglycerides were separated by effective carbon number (ECN). The primary objective was to determine the degree of saturation for each triacylglyceride by the combination of ECN and mass (see Table 5).

In this study, the data showed that the triacylglyceride fraction containing one single bond was dominant. Predominantly saturated triacylglycerides and predominantly unsaturated triacylglycerides were combined in groups as described in Example 1. The ratios of the groups predominantly saturated triacylglycerides, SATs, to predominantly unsaturated triacylglycerides, UNSATs were compared. The SATs/UNSATs ratio of the triacylglycerides in the feces of the treatment group fed cheese diet comprising 4 weight percent HPMC was 1.0, whereas the SATs/UNSATs ratio of the triacylglycerides in the feces of the control group fed cheese diet comprising 4 weight percent MCC was 0.78. These results show a trend of an increased SATs/UNSATs ratio of the triacylglycerides in the feces from the cheese fed treatment group, as compared to the SATs/UNSATs ratio of the triacylglycerides in the feces from the cheese fed control group, although the difference was not statistically significant. These results were not surprising, because there appears to be a strong relationship between the total fat content of a given food relative to the preferential excretion of saturated triacylglycerides. In this study, cheese had a total fat content of less than 15 percent.

Collectively, this study shows that water-soluble cellulose derivatives, such as hydroxypropyl methylcellulose, have the ability to preferentially interact with saturated triacylglcyerides in a high fat diet, but that fat composition is important in whether or not HPMC can more preferentially interact with saturated triacylglcyerides than MCC.

### Example 4

An animal study was conducted with male Golden Syrian hamsters with a starting body weight of between 80-90 grams (LVG strain, Charles River Laboratory, Willmington, Mass.) in each of the diets specified below. The animal study was approved by the Animal Care and Use Committee, Western Regional Research Center, USDA, Albany, Calif. The male Syrian Golden hamsters were divided into two main groups. One group was called the “treatment group” and was fed a diet comprising hydroxypropyl methylcellulose (HPMC), a water-soluble cellulose ether. The other group was called the “control group” and was fed a diet comprising microcrystalline cellulose (MCC). Each group consisted of approximately 10 hamsters.

Treatment Group: “Bologna” Diet, 4 weight percent HPMC

This treatment group was fed a “bologna” diet consisting of the following: 33 g bologna (freeze dried and powdered), 10 g of casein, 0.3 g of DL-methionine, 45.4 g of cornstarch, 2.5 g of corn oil, 0.3 g of choline biturate, 1 g of a vitamin mixture, 3.5 g of a mineral mixture, and 4 g of the same hydroxypropyl methylcellulose as in Example 1.

Control Group: “Bologna” Diet, 4 weight percent MCC

This treatment group was fed a “bologna” diet consisting of the following: 33 g bologna (freeze dried and powdered), 10 g of casein, 0.3 g of DL-methionine, 45.4 g of cornstarch, 2.5 g of corn oil, 0.3 g of choline biturate, 1 g of a vitamin mixture, 3.5 g of a mineral mixture, and 4 g of microcrystalline cellulose.

All hamsters had been fed laboratory standard diet (lab “chow”) for seven days to acclimate. At the end of a 7 day period, feces were sampled for 2 days from all animals During the feeding of the specified diet as described above feces were collected from each hamster after 6 days. Samples were freeze dried at each collection time.

### Analysis of Bile Acids, Sterols, and Acylglycerides in Hamster Feces Fed Bologna

The method for analysis of hamster fecal samples for the determination of bile acids, sterols, mono-, di-, and tri-acylglycerides was done by HPLC as previously described in Example 1.

### Results

Hamsters were fed a chow diet for seven days, and then changed to a celluosic-supplemented diet (bologna) for 6 days. Fecal lipid levels were determined and summarized in Table 10. The data was analyzed using JMP statistical software using One Way Analysis of Variance (ANOVA) and the means tested using the Student's t-Test.

TABLE 10
Summary of average analyte class concentration
for bologna (B) diet supplemented with either HPMC or MCC

| | | Average mg/g feces | | | | |
|---|---|---|---|---|---|---|
| | | Bile | | | | |
| Diet | Day | Acids | FFAs/MAGs | Sterols | DAGs | TAGs |
| Chow | 0 | 4.02 | 19.09 | 10.94 | 4.59 | 2.25 |
| B/MCC | 6 | 8.71 | 157.27 | 19.60 | 7.25 | 1.62 |
| B/HPMC | 6 | 34.43 | 269.87 | 22.24 | 8.89 | 3.37 |

The bologna diet supplemented with HPMC showed a significant increase (p<0.05) in bile acids, free fatty acids/monoglycerides (FFAs/MAGs), and triacylglycerides compared to bologna diet supplemented with MCC. While not statistically significant (p<0.05) the bologna diet supplemented with HPMC showed an increase in both sterols and diacylglycerides compared to the cheese diet supplemented with MCC. Table 10 that illustrates water-soluble cellulose derivatives are useful for reducing the amount of saturated fatty acids capable of being absorbed by an animal body relative to the amount of unsaturated fatty acids capable of being absorbed by an animal body after the consumption of a fat-containing food product. The analysis of fecal bile acids illustrates that water-soluble cellulose derivatives, such as hydroxypropyl methylcellulose facilitate the excretion of bile acids as well as cholesterol-derived metabolites in the feces of hamsters. Thus, water-soluble cellulose derivatives, such as hydroxypropyl methylcellulose, may be used as gastrointestinally active biological agents in the removal of lipids.

### Analysis of Total Saturated Fatty Acids and Unsaturated Fatty Acids in Feces of Hamsters Fed Bologna

The method for analysis of hamster fecal samples for the determination of total saturated fatty acids (SATs) and total unsaturated fatty acids (UNSATs) were done by FAME analysis as previously described in Example 1.

### Results

To further illustrate the selectivity of hydroxypropyl methylcellulose on lipid levels, the ratio of SATs/UNSATs was determined by comparing the starting food composition to the feces. The data was analyzed using JMP statistical software. Within each group the levels of species of interest were analyzed with JMP using Means ANOVA Pooled t-Test. The food composition and the feces analysis of the summation of saturated and unsaturated fatty acids are presented in Table 11.

TABLE 11
Summary of total saturated and unsaturated fatty acids from bologna
diet feces collected at day 6 from hamsters fed the bologna diet

| | | FAME | | |
|---|---|---|---|---|
| | FAME SATs | UNSATs | | Total Fats |
| | Sum mg/g | Sum mg/g | | [SATs + |
| | AVG* all | AVG* all | SATs/UNSATs | UNSATs] |
| Day/Additive | animals | animals | Ratio | mg/g |
| Food | 77 | 105 | 0.7 | 182 |
| Composition | | | | |
| Day 6 Feces, | 218 | 50 | 4.4 | 268 |
| HPMC | | | | |

*Average

Hamsters fed the bologna diet supplemented with 4% hydroxypropyl methylcellulose had significantly higher SATs/UNSATs ratio in the feces compared to the SATs/UNSATs ratio in the starting bologna diet. Thus, hydroxypropyl methylcellulose significantly altered the ratio of total saturated fatty acids relative to unsaturated fatty acids when comparing the SATs/UNSATs ratios of the starting food composition relative to the excreted feces. These observations correlate with the bile acids, sterols, free fatty acids, monoacylglycerides, diacylglycerides, and triacylglycerides. Overall the HPMC facilitated the excretion of the saturated fatty acids from hamsters fed a defined bologna diet. Table 9 illustrates that water-soluble cellulose derivatives are useful for reducing the amount of saturated fatty acids capable of being absorbed by an animal body relative to the amount of unsaturated fatty acids capable of being absorbed by an animal body after the consumption of a fat-containing food product.

Analysis of Triacylglycerides in Feces from Hamsters Fed a Bologna Diet

In order to determine if a water-soluble cellulose derivative, such as hydroxypropyl methylcellulose, increased the selectivity of the fatty acid profile of the excreted lipids, hamster feces were analyzed by HPLC coupled with a mass spectrometry as described in Example 1. Again triacylglycerides were separated by effective carbon number (ECN). The primary objective was to determine the degree of saturation for each triacylglyceride by the combination of ECN and mass (see Table 5).

In this study, the data showed that the triacylglyceride fraction containing one single bond was dominant. Predominantly saturated triacylglycerides and predominantly unsaturated triacylglycerides were combined in groups as described in Example 1. The ratios of the groups predominantly saturated triacylglycerides, SATs, to predominantly unsaturated triacylglycerides, UNSATs were compared. The SATs/UNSATs ratio of the triacylglycerides in the feces of the treatment group fed Bologna diet comprising 4 weight percent HPMC was 0.72, whereas the SATs/UNSATs ratio of the triacylglycerides in the feces of the control group fed Bologna diet comprising 4 weight percent MCC was 0.62.

These results show a trend of an increased SATs/UNSATs ratio of the triacylglycerides in the feces from the Bologna fed treatment group, as compared to the SATs/UNSATs ratio of the triacylglycerides in the feces from the Bologna fed control group, although the difference was not statistically significant. Again these results were not surprising, because there appears to be a strong relationship between the total fat content of a given food relative to the preferential excretion of saturated triacylglycerides. In this study, Bologna had a total fat content of less than 19 percent.

Collectively, this study shows that water-soluble cellulose derivatives, such as hydroxypropyl methylcellulose, have the ability to preferentially interact with saturated triacylglcyerides in a high fat diet, but that fat composition is important in whether or not HPMC can more preferentially interact with saturated triacylglcyerides than MCC.

What is claimed is:

1. A food product comprising
a) a fat rich in one or more saturated fatty acids,
b) a fat rich in one or more unsaturated fatty acids, and
c) at least 2 weight percent of one or more water-soluble cellulose derivatives, based on the total weight of the food product.

2. The food product of claim 1 wherein the food product comprises at least 5% weight percent of a fat rich in one or more saturated fatty acids.

3. The food product of claim 1 wherein the food product comprises at least 5% weight percent of a fat rich in one or more predominantly saturated triacylglycerides.

4. The food product of claim 1 wherein the weight ratio between a) the fat rich in one or more saturated fatty acids and b) the fat rich in one or more unsaturated fatty acids is from 0.05 to 15:1.

5. The food product of claim 1 wherein the fat rich in one or more saturated fatty acids is selected from the group consisting of palm oil, palm kernel oil, coconut oil, cacao butter, fat of milk and milk products, lard, tallow, fat of meat and meat products.

6. The food product of claim 1 wherein the fat rich in one or more unsaturated fatty acids is selected from the group consisting of cottonseed oil, wheat germ oil, tea seed oil, soy oil, olive oil, peanut oil, corn oil, sunflower oil, safflower oil, rapeseed oil, canola oil, grape seed oil, sesame oil, flax seed oil, walnut oil, oils in avocados, or fish oils.

7. The food product of claim 1 wherein the water-soluble cellulose derivative is a water-soluble cellulose ether or cellulose ester.

8. The food product of claim 1 being a processed meat product, a dairy product, a fried product, a product able to be fried, a sauce, a bakeable or baked product or a snack bar.

9. A food product in the form of a processed meat product, a dairy product, a fried product or a product able to be fried comprising
i) one or more saturated fatty acids,
ii) one or more unsaturated fatty acids, and
iii) at least 2 weight percent of one or more water-soluble cellulose derivatives, based on the total weight of the food product.

10. The food product of claim 9 comprising at least 5 weight percent of one or more saturated fatty acids.

11. The food product of claim 9 comprising at least 5 weight percent of one or more predominantly saturated triacylglycerides.

12. The food product of claim 9 wherein the water-soluble cellulose derivative is a water-soluble cellulose ether or cellulose ester.

13. A method of reducing the amount of saturated fatty acids capable of being absorbed by an animal body relative to the amount of unsaturated fatty acids capable of being absorbed by an animal body in a fat-containing food product comprising the steps of
i) identifying a food product comprising one or more saturated fatty acids and one or more unsaturated fatty acids, and
ii) incorporating one or more water-soluble cellulose derivatives in the food product.

14. The method of claim 13 wherein the [(weight saturated fatty acids in food product) divided by (weight unsaturated fatty acids in food product)]=[(m)×(weight saturated fatty acids capable of being absorbed by animal body) divided by (weight unsaturated fatty acids capable of being absorbed by animal body)], wherein m is at least 1.5.

15. A method of reducing the amount of saturated fatty acids capable of being absorbed by an animal body relative to the amount of unsaturated fatty acids capable of being absorbed by an animal body from a fat-containing food product comprising the steps of
i) identifying a food product comprising one or more saturated fatty acids and one or more unsaturated fatty acids, and
ii) administering one or more water-soluble cellulose derivatives before, during or after the consumption of the food product.

16. The method of claim 15 wherein the [(weight saturated fatty acids in food product) divided by (weight unsaturated fatty acids in food product)]=[(m)×(weight saturated fatty acids capable of being absorbed by animal body) divided by (weight unsaturated fatty acids capable of being absorbed by animal body)], wherein m is at least 1.5.

17. A method of reducing the effective caloric content contributed by one or more saturated fatty acids relative to the caloric content contributed by one or more unsaturated fatty acids in a fat-containing food product comprising the steps of
i) identifying a food product comprising one or more saturated fatty acids and one or more unsaturated fatty acids, and
ii) incorporating one or more water-soluble cellulose derivatives in the food product.

18. The method of claim 17 wherein the effective caloric content contributed by one or more saturated fatty acids relative to the caloric content contributed by one or more unsaturated fatty acids is reduced by at least 5 percent by incorporating one or more water-soluble cellulose derivatives in the food product.

19. A method of reducing the effective caloric content contributed by one or more saturated fatty acids relative to the caloric content contributed by one or more unsaturated fatty acids from a fat-containing food product comprising the steps of
i) identifying a food product comprising one or more saturated fatty acids and one or more unsaturated fatty acids, and
ii) administering one or more water-soluble cellulose derivatives before, during or after the consumption of the food product.

20. The method of claim 19 wherein the effective caloric content contributed by one or more saturated fatty acids relative to the caloric content contributed by one or more unsaturated fatty acids is reduced by at least 5 percent by administering one or more water-soluble cellulose derivatives before, during or after the consumption of the food product.

21. A method of reducing the amount of predominantly saturated triacylglycerides capable of being hydrolyzed in the intestine by lipases relative to the amount of predominantly unsaturated triacylglycerides capable of being hydrolyzed in the intestine by lipases in a fat-containing food product comprising the steps of
i) identifying a food product comprising one or more predominantly saturated triacylglycerides and one or more predominantly unsaturated triacylglycerides, and
ii) incorporating one or more water-soluble cellulose derivatives in the food product.

22. The method of claim 21 wherein the total fat content in the food product is at least 20 weight percent.

23. A method of reducing the amount of predominantly saturated triacylglycerides capable of being hydrolyzed in the intestine by lipases relative to the amount of predominantly unsaturated triacylglycerides capable of being hydrolyzed in the intestine by lipases from a fat-containing food product comprising the steps of
i) identifying a food product comprising one or more predominantly saturated triacylglycerides and one or more predominantly unsaturated triacylglycerides, and
ii) administering one or more water-soluble cellulose derivatives before, during or after the consumption of the food product.

24. The method of claim 23 wherein the total fat content in the food product is at least 20 weight percent.
